(19) United States
(12) Patent Application Publication
(10) Pub. No.: US 2011/0247496 A1
(43) Pub. Date: Oct. 13, 2011

(54) METHOD OF HUMIDIFYING A GAS STREAM AND ASSEMBLY THEREFOR

(75) Inventors: Kimm Gardner, Carlsbad, CA (US); Mabini Arcilla, San Diego, CA (US)

(73) Assignee: KONINKLIJKE PHILIPS ELECTRONICS N.V., EINDHOVEN (NL)

(21) Appl. No.: 13/139,753

(22) PCT Filed: Dec. 4, 2009
(86) PCT No.: PCT/IB09/55527

§ 371 Date: Jun. 15, 2011

Related U.S. Application Data
(60) Provisional application No. 61/139,664, filed on Dec. 22, 2008.

(51) Int. Cl. A61M 16/16; F24F 6/00

(52) U.S. Cl. 95/210; 261/152; 261/128; 95/214; 96/294

(57) ABSTRACT

The present invention provides a humidifier assembly for humidifying a gas stream. The humidifier assembly including a heating element structured to be disposed in the gas stream for directly heating the gas stream as the gas stream passes therethrough and a supply of fluid provided at or near the heating element.

### FIELD OF THE INVENTION

The invention relates generally to humidifier assemblies for humidifying a gas stream and, more particularly, to humidifier assemblies having an integrated heat source. The invention also relates to methods of humidifying a gas stream.

### BACKGROUND OF THE INVENTION

The higher incidence and awareness of airborne diseases (e.g., SARS, Avian Flu) have made the filtration of exhaled gas for patients on mechanical ventilation a requirement in some healthcare environments. Exhaled gases can commonly reach a ventilator unit at 37 C and near 100% relative humidity. Accordingly, in-line filters used in such applications need to be able to withstand moisture. The administration of inline aerosol medications can also result in the filter elements being similarly subjected to moisture. Wetting of filter elements can weaken and lead to breaches in the filter media of such filter elements. Wet filter elements also have higher flow resistance which can make it more difficult for the patient to force air through a filter element (through exhalation or inhalation).

Another concern is condensation formation in the ducting or tubing associated with ventilation devices and the negative effects of such condensation on components (e.g., without limitation, flow sensors) downstream of a filter element. In order to avoid such condensation, it is desirable to raise the temperature of the gases flowing within the ventilation devices.

As such, it is desirable to place a bacteria filter in the exhalation gas pathway that can maintain its integrity and low flow resistance when subjected to moisture, while simultaneously increasing the temperature of the exiting gas to prevent rainout within the ventilation device

Known devices heat the filter housing in attempting to keep the filter elements dry and increase the temperature of the exhaled gases. Such devices are generally inefficient, as a portion of the energy applied is typically lost to the surrounding environment rather than being used to heat the media and gas. Because the increase in gas temperature is limited due to such losses, condensation can still occur downstream of the filter. Additionally, known devices often allow moisture to collect on the filter media.

There is thus room for improvement in the area of filters and filter assemblies used in filtering of exhalation gases.

### SUMMARY OF THE INVENTION

In one embodiment, the invention provides a humidifier assembly for humidifying a gas stream. The humidifier assembly comprises a heating element structured to be disposed in the gas stream for directly heating the gas stream as the gas stream passes therethrough and a supply of fluid provided at or near the heating element. The heating element may comprise a porous structure that is structured to accommodate the gas stream passing therethrough. The heating element may be arranged in a generally planar structure. The heating element may be generally thin and flexible and arranged in a generally non-planar structure. The heating element may comprise a carbon fiber heating element. The fluid may comprise water. The fluid may comprise a medicinal fluid. The gas stream may comprise inhalation gases. The fluid may be provided to a filter element disposed in direct contact with, or in close proximity to the heating element.

Another embodiment provides a method of humidifying a gas stream. The method comprises directly heating the gas stream as the gas stream passes through a heating element and providing a fluid supply at or near the heating element. The heating element may comprise a porous structure that accommodates the gas stream passing therethrough. The heating element may be structured to absorb portions of the fluid. The heating element may be arranged in a generally planar structure. The heating element may be generally thin and flexible and arranged in a generally non-planar structure. The heating element may comprise a carbon fiber heating element. The fluid may comprise water. The fluid may comprise a medicinal fluid. The method may further comprise prior to directly heating the gas stream, filtering the gas stream by passing the gas stream through a filter element. The gas stream may comprise inhalation gases.

A further embodiment provides a method of humidifying a gas stream. The method comprises providing a heating element disposed in the gas stream, providing a filter element disposed in the gas stream, the filter element being disposed in direct contact with, or in close proximity to, the heating element, providing moisture to the filter element, and directing the gas stream through the heating element and the filter element.

### BRIEF DESCRIPTION OF THE DRAWINGS

The accompanying drawings illustrate presently preferred embodiments of the invention, and together with the general description given above and the detailed description given below, serve to explain the principles of the invention. As shown throughout the drawings, like reference numerals designate like or corresponding parts.

FIG. 1 is a simplified cross-sectional view of a filter assembly in accordance with an embodiment of the invention;

FIG. 2 is a simplified cross-sectional view of a filter assembly in accordance with another embodiment of the invention;

FIG. 3 is a simplified cross-sectional view of a filter assembly in accordance with yet another embodiment of the invention; and

FIG. 4 is a simplified cross-sectional view of a filter assembly in accordance with a further embodiment of the invention.

FIG. 5 is a simplified cross-sectional view of a filter assembly in accordance with a another further embodiment of the invention.

### DESCRIPTION OF THE PREFERRED EMBODIMENTS

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed herein, the statement that two or more parts or components are “coupled” together shall mean that the parts are joined or operate together either directly or through one or more intermediate parts or components.

As employed herein, the term “number” shall mean one or an integer greater than one (i.e., a plurality).

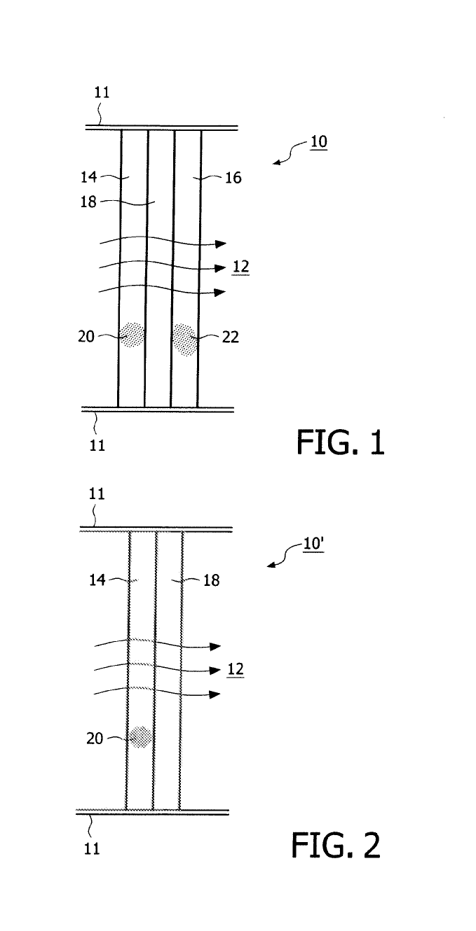

FIG. 1 shows a cross-sectional view of a filter assembly 10 according to an embodiment of the invention. Filter assembly 10 is housed within a conduit 11 or other suitable structure (e.g., without limitation, rigid plastic housing, flexible or rigid tubing) for directing a gas stream 12 passing therethrough. Gas stream 12 may comprise, for example, without limitation, inhalation gases for, or exhalation gases from, a medical patient. Filter assembly 10 is positioned in the gas stream 12 in order to provide filtration of the gas stream 12 passing therethrough. The filter assembly 10 includes a first filter element 14 and a second filter element 16 and a heating element 18 disposed therebetween. Each of the filter elements 14,16 are of generally planar structure and comprise suitable filter media 20, 22 for filtering of the gas stream 12. Examples of such filter media include, without limitation, paper, porous cellulose, ceramic microfiber, electrostatically treated polypropylene. Although the embodiment depicted in FIG. 1 shows the heating element 18 in direct contact with both the first filter element 14 and the second filter element 16, it is to be appreciated that one or both of the number of filter elements 14,16 may be spaced apart from the heating element 18 without departing from the scope of the invention. While direct contact provides the best transfer of heat from the heater to the filter element, it is to be appreciated that an acceptable effect can also be achieved if there is a space between the two components and sufficient power is provided to the heater.

Heating element 18 preferably comprises a thin, sheet-like, generally flexible, porous structure such as, without limitation, a carbon fiber heating element. A carbon fiber heating element resembles a sheet of fabric, and can be woven such that it is porous to gas flow. The carbon fibers of heating element 18 are electrically conductive and generate heat when a voltage differential is placed across them.

The structure of heating element 18 allows it to be disposed directly in gas stream 12. Additionally, such structure also allows for the heating element 18 to be disposed in close proximity to, and preferably in direct contact with, the filter media 20, 22 of the first and second filter elements 14, 16, and thus provide heat directly to the filter media 20, 22. Thus, when humid gases flow through the filter assembly 10, as shown by arrows 12 in FIG. 1, the heater element 18 prevents moisture from humid exhaled gases from accumulating in the filter media 20, 22. Keeping the media dry prevents undesirable increases in flow resistance across the filter assembly 10 while also helping to reduce filter breakdown and deterioration. The heater element 18 also increases the temperature of the humid gas stream 12, thereby preventing condensation as the humid gas 12 exits the filter assembly 10 and comes into contact with ventilator components (not shown) downstream.

It is to be appreciated that placement of the heating element 18 directly in the gas stream 12 provides for more uniform heating of the gas stream 12 than known designs that heat the housing through which the gas stream passes, thus not uniformly heating the gas. Additionally, such placement of the heating element 18 within the gas stream 12 more efficiently utilizes the heating element 18 in transferring heat energy to the gas stream 12 instead of the conduit 11 and/or related housings (not shown).

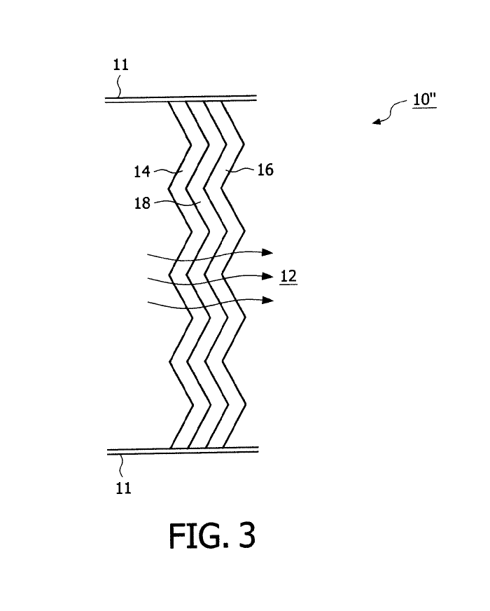

The design of filter assembly 10 may be varied in order to provide optimum filtration properties for varied conditions. For example, FIG. 2 shows a filter assembly 10′ that is similar to that previously discussed in connection with FIG. 1. However, as seen in FIG. 2, the filter assembly 10′ includes only two components instead of three, namely a heating element 18 and a first filter element 14. FIG. 3 shows an example filter assembly 10″ that, like the example of FIG. 1, includes 3 components, a first filter element 14, a second filter element 16, and a heating element 18 disposed therebetween. However, unlike the example shown in FIG. 1, filter elements 14 and 16 have been formed into a generally corrugated, or pleated, structure in order to increase the filtration area of the filter assembly 10″. As shown in FIG. 3, the generally flexible nature of heating element 18 allows for it to also be formed into such a corrugated or pleated structure together with the filter elements 14, 16. Accordingly, it is to be appreciated that the filter assembly of the present invention may be varied according to specific needs in desired applications (e.g., without limitation, varying the quantity and/or arrangement of filter elements and/or heating elements, varying the dimensions and/or composition of filter elements and/or heating elements) without departing from the scope of the present invention.

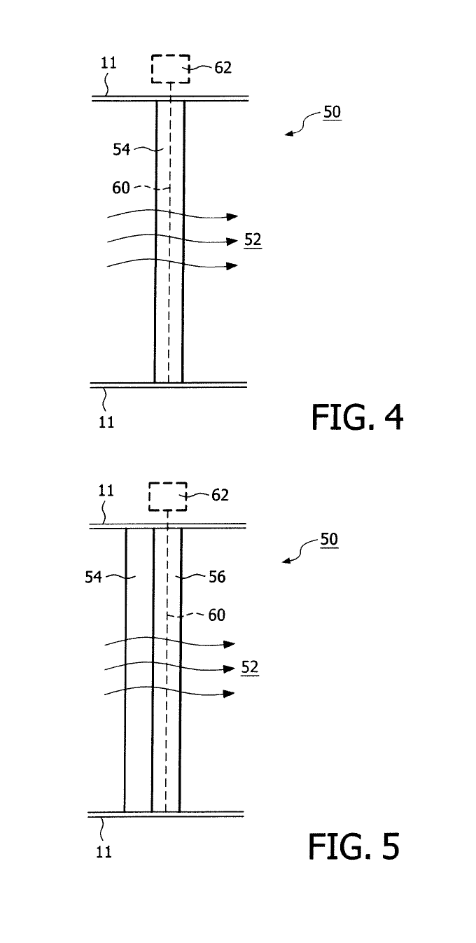

FIG. 4 shows cross-sectional view of a humidifier assembly 50 according to another embodiment of the invention. Humidifier assembly 50 is disposed within a conduit 11 or other suitable structure for directing a gas stream 52 passing therethrough. Gas stream 52 may comprise, for example, a gas supply for inhalation by a user (e.g., without limitation, air, air/oxygen mixture, air/oxygen mixture and anesthetic agents, helium/air/oxygen). Humidifier assembly 50 is positioned in the gas stream 52 in order to provide heat and humidity to the gas stream 52 as the gas stream 52 passes therethrough. Humidifier assembly 50 includes a heating element 54 disposed in the gas stream 52. Like heating element 18, previously discussed, heating element 54 preferably comprises a thin, sheet-like, generally flexible, porous structure such as, without limitation, a carbon fiber heating element. Such structure allows for the heating element 54 to be formed in a planar structure, such as shown in FIG. 4, or into non-planar structures (not shown) as desired to meet requirements for varied applications. Unlike the filter assemblies 10, 10′ and 10″, humidifier assembly 50 further includes a fluid supply 60 provided at or near the heating element 54. Preferably, fluid supply 60 is provided in a manner (e.g., without limitation, spray, stream, drops) such that it is uniformly absorbed by the heating element 54. Such absorbed fluid can then evaporate into the heated air passing through the heating element 54. Fluid supply 60 may be comprised of, for example, without limitation, water or medication in liquid form that may be supplied by a reservoir 62 or other suitable source. Through the interaction of the fluid supply 60 and the heating element 54, the humidifier assembly 50 can be used to provide heated and humidified inspiratory air to a medical patient. Preferably, the combination of the porosity of the heating element 54 and the rate at which fluid supply 60 is provided is adequate to add moisture to the gas stream 52 without noticeably increasing the resistance to flow through the humidifier assembly 50.

FIG. 5 shows another embodiment of the humidifier assembly 50 employed in conjunction with a hydrophilic filter element 56 positioned in contact with, or in close proximity to, the heating element 54. In this embodiment, the fluid supply 60 is provided to the hydrophilic filter element 56 such that the hydrophilic filter element 56 absorbs the fluid supply 60 from the humidifier assembly 50. It can be readily appreciated that such an arrangement could be employed to provide filtered, heated and humidified inspiratory air to a medical patient.

It is to be appreciated that although the fluid supply 60 is shown in FIGS. 4 and 5 as being supplied to the heating element 54 or filter element 56, the fluid supply 60 could be supplied near the heating element 54 or filter element 56, and not actually to, without departing from the scope of the invention.

While preferred embodiments of the invention have been described and illustrated above, it should be understood that these are exemplary of the invention and are not to be considered as limiting. Additions, deletions, substitutions, and other modifications can be made without departing from the spirit or scope of the present invention. Accordingly, the invention is not to be considered as limited by the foregoing description but is only limited by the scope of the appended claims.

What is claimed is:

1. A humidifier assembly for humidifying a gas stream, comprising:
a heating element structured to be disposed in the gas stream for directly heating the gas stream as the gas stream passes therethrough; and
a supply of fluid provided at or near the heating element.

2. The humidifier assembly of claim 1 wherein the heating element comprises a porous structure that is structured to accommodate the gas stream passing therethrough.

3. The humidifier assembly of claim 2 wherein the heating element is arranged in a generally planar structure.

4. The humidifier assembly of claim 2 wherein the heating element is generally thin and flexible and arranged in a generally non-planar structure.

5. The humidifier assembly of claim 4 wherein the heating element comprises a carbon fiber heating element.

6. The humidifier assembly of claim 1 wherein the fluid comprises water.

7. The humidifier assembly of claim 1 wherein the fluid comprises a medicinal fluid.

8. The humidifier assembly of claim 1 wherein the gas stream comprises inhalation gases.

9. The humidifier assembly of claim 1 wherein the fluid is provided to a filter element disposed in direct contact with, or in close proximity to the heating element.

10. A method of humidifying a gas stream, comprising:
directly heating the gas stream as the gas stream passes through a heating element;
providing a fluid supply at or near the heating element.

11. The method of claim 10 wherein the heating element comprises a porous structure that accommodates the gas stream passing therethrough.

12. The method of claim 10 wherein the heating element is structured to absorb portions of the fluid.

13. The method of claim 11 wherein the heating element is arranged in a generally planar structure.

14. The method of claim 11 wherein the heating element is generally thin and flexible and arranged in a generally non-planar structure.

15. The method of claim 10 wherein the heating element comprises a carbon fiber heating element.

16. The method of claim 10 wherein the fluid comprises water.

17. The method of claim 10 wherein the fluid comprises a medicinal fluid.

18. The method of claim 10 further comprising prior to directly heating the gas stream, filtering the gas stream by passing the gas stream through a filter element.

19. The method of claim 10 wherein the gas stream comprises inhalation gases.

20. A method of humidifying a gas stream, comprising:
providing a heating element disposed in the gas stream;
providing a filter element disposed in the gas stream, the filter element being disposed in direct contact with, or in close proximity to, the heating element;
providing moisture to the filter element; and
directing the gas stream through the heating element and the filter element.
